(19) United States
(12) Patent Application Publication
(10) Pub. No.: US 2022/0233612 A1
(43) Pub. Date: Jul. 28, 2022

(54) PROBIOTICS TO INHIBIT ENTERIC PATHOGENS

(71) Applicant: SOUTH DAKOTA BOARD OF REGENTS TECHNOLOGY TRANSFER OFFICE, Brookings, SD (US)

(72) Inventor: Joy Scaria, Brookings, SD (US)

(21) Appl. No.: 17/615,556

(22) PCT Filed: May 30, 2020
(86) PCT No.: PCT/US2020/035468

§ 371 Date: Nov. 30, 2021

Related U.S. Application Data
(60) Provisional application No. 62/855,586, filed on May 31, 2019.

(51) Int. Cl. A61K 35/747; C12N 1/20; C12Q 1/18; A61K 35/744; A61K 35/742; A61K 35/741; A61P 31/04

(52) U.S. Cl. CPC A61K 35/747; C12N 1/20; C12Q 1/18; A61P 31/04; A61K 35/742; A61K 35/741; A61K 35/744

(57) ABSTRACT

A method for identifying a microbial composition that inhibits colonization of an enteric pathogen of a first animal is disclosed. The method includes removing a microbial sample from the digestive tract of a second animal. The method further includes culturing the microbial sample, isolating, cultivating and identifying microbial species within the microbial sample. The method further includes creating compositions of one or more isolated microbial species and determining the ability of the composition to inhibit growth of an enteric pathogen in at least one of an in vitro or an in vivo assay. The method further includes identifying a microbial composition capable of inhibiting growth of enteric pathogens in a first animal. A microbial composition that inhibits colonization of an enteric pathogen of a first animal is also disclosed. The composition includes microorganisms from a group comprising Faecalicoccus, Lactobacillus, Megamonas, Staphylococcus, Bacillus, Enterococcus, Olsenella, Megasphaera, Pseudoflavonifractor, and Massiliomicrobiota.

### CROSS-REFERENCE TO RELATED APPLICATIONS

The present application is a 371 U.S. National Phase of International Application No. PCT/US2020/035468, filed May 30, 2020, which claims priority to U.S. Provisional Application Ser. No. 62/855,586 filed May 31, 2019. International Application number PCT/US2020/035468 and U.S. Provisional Application Ser. No. 62/855,586 are hereby incorporated by reference in the entirety.

### TECHNICAL FIELD

The present invention generally relates to probiotics, and in particular, probiotics for preventing disease in domesticated animals.

### BACKGROUND

A dense and complex microbial community colonizes the human and animal gastrointestinal tract over time. This complex community, collectively called the gut microbiota, provides a range of functions such as the development of the immune system, digestion, tissue integrity, vitamin and nutrient production, and the ability to prevent colonization of enteric pathogens. With the advances in microbiome research and because of the worldwide increase in bacterial antibiotic resistance, there is high interest in using mature gut microbiome as an alternative means of suppressing enteric infections. The ability of the healthy gut microbiota to prevent pathogen colonization has been demonstrated in poultry, in which inoculation of young chickens with adult chicken feces prevented the colonization of Salmonella. The same concept was used in recent years to treat recurrent Clostridium difficile infection in humans by fecal transplantation from healthy individuals.

Although colonization resistance of the gut microbiota was first demonstrated in poultry, Salmonella colonization in poultry continues to be a significant problem. Poultry has been identified as the most common food in outbreaks with pathogens in the United States. The poultry industry has responded to this problem by implementing biosecurity measures that are designed to minimize exposure to pathogens by the chicken. However, increased biosecurity and clean conditions in the production system would also decrease the exposure to commensal bacteria and would reduce the microbiome diversity in the chicken gut. One proposed hypothesis is that reduced exposure to commensal gut microbes would open nutrient niches in the gut that can be easily used by pathogens which increases their colonization risk. To reduce this risk, the poultry industry has attempted to reduce the pathogen colonization by inoculating chicken with complex commensal bacterial blends such as the lyophilized mixture of anaerobic bacteria from the cecum of adult chicken, bacteria from healthy chicken mucosal scrapings, and continuous flow cultures of cecal chicken bacteria. However, due to the complexity of these mixtures, it is difficult to understand their mechanism of action and improve their efficacy. Therefore, it would be advantageous to provide a system, method, and composition that overcomes the shortcomings described above.

### SUMMARY

A method for identifying a microbial composition that inhibits colonization of an enteric pathogen in at least one first animal is disclosed. In embodiments, the method includes removing a microbial sample from a digestive tract of at least one second animal. In embodiments, the method further includes culturing the microbial sample. In embodiments, the method further includes isolating a microbial species within a cultivated microbial sample. In embodiments, the method further includes identifying the microbial species. In embodiments, the method further includes creating compositions of one or more isolated microbial species. In embodiments, the method further includes determining an ability of the compositions to inhibit growth of an enteric pathogen in at least one of an in vitro or an in vivo assay. In embodiments, the method further includes identifying a microbial composition capable of inhibiting growth of enteric pathogens in the at least one first animal.

In embodiments of the method, the method further includes administering the microbial composition to one or more animals to inhibit growth of enteric pathogens.

A microbial composition that inhibits colonization of an enteric pathogen in at least one first animal, prepared by process, is also disclosed. In embodiments, the process includes removing a microbial sample from a digestive tract of at least one second animal. In embodiments, the process further includes culturing the microbial sample. In embodiments, the process further includes isolating a microbial species within a cultivated microbial sample. In embodiments, the process further includes identifying the microbial species. In embodiments, the process further includes creating a composition of at least one or more isolated microbial species. In embodiments, the process further includes determining an ability of the composition to inhibit growth of an enteric pathogen in at least one of an in vitro or in vivo assay. In embodiments, the process further includes identifying a microbial composition capable of inhibiting growth of enteric pathogens in the at least one first animal. In embodiments, the process further includes fashioning the microbial composition into a form capable of enteric administration.

A method of administering a microbial composition that inhibits colonization of an enteric pathogen to one or more of an at least one first animals is also disclosed. In embodiments, the microbial composition includes identifying the at least one first animal with an at least one of an active enteric disease or risk of enteric disease. In embodiments, the microbial composition further includes administering to the at least one or more of the at least one first animal a microbial composition comprised of a mixture of at least one of a microbial isolate, isolated from an at least one of a second animal, wherein the microbial composition is administered enterically.

A microbial composition that inhibits colonization of an enteric pathogen in at least one animal is also disclosed. In embodiments, the microbial composition includes a therapeutically effective amount of a plurality of viable microorganisms from two or more genera selected from a group consisting of Faecalicoccus, Lactobacillus, Megamonas, Staphylococcus, Bacillus, Enterococcus, Olsenella, Megasphaera, Pseudo flavonifractor, and Massiliomicrobiota.

In embodiments of the microbial composition, the plurality of viable microorganisms further includes two or more species or genera selected from the group consisting of Faecalicoccus pleomorphus, Lactobacillus agilis, Staphylococcus saprophyticus, Bacillus paralicheniformis, Megamonas funiformus Enterococcus durans, Megasphaera statonii, Massiliomicrobiota timonensis, Olsenella, and Pseudo flavonifractor.

### BRIEF DESCRIPTION OF THE DRAWINGS

The numerous advantages of the disclosure may be better understood by those skilled in the art by reference to the accompanying figures in which:

FIG. 1 is a flow diagram illustrating a method for identifying a microbial composition that inhibits the colonization of enteric infections in a first animal, in accordance with one or more embodiments of the present disclosure;

FIG. 2 is a flow diagram illustrating a method of administering a microbial composition that inhibits colonization of an enteric pathogen in animals, in accordance with one or more embodiments of the present disclosure; FIG. 3 is a chart illustrating an overview of the culture conditions as well as diversity and frequency of isolated microbial species in an example microbial composition, in accordance with one or more embodiments of the present disclosure;

FIG. 4 is a graph illustrating microbial species that show varying degrees of inhibition against S. Typhimurium, in accordance with one or more embodiments of the present disclosure;

FIG. 5A is a graph illustrating the effectiveness of various microbial blends for S. Typhimurium inhibition, in accordance with one or more embodiments of the present disclosure;

FIG. 5B illustrates a table describing the bacterial strains used to formulate

MIX10, in accordance with one or more embodiments of the present disclosure.

FIG. 6 is a chart illustrating a detailed timeline for testing microbial blends for S. Typhimurium inhibition in vivo, in accordance with one or more embodiments of the present disclosure;

FIG. 7 is a graph illustrating the inhibition of a microbial blend on S. Typhimurium in vivo, in accordance with one or more embodiments of the present disclosure;

FIG. 8 is a photograph of transverse sections of bird cecums illustrating the effect of microbial compositions on the intestine of an animal infected with S. Typhimurium, in accordance with one or more embodiments of the present disclosure;

FIG. 9 is a graph illustrating the effect of a microbial composition on the intestine of an animal infected with S. Typhimurium, in accordance with one or more embodiments of the present disclosure;

FIG. 10 is a graph illustrating an mRNA profile of pooled cecal tissue for various inflammatory cytokines, chemokines, and other genes under various conditions, in accordance with one or more embodiments of the present disclosure;

FIG. 11 is a graph illustrating the relative abundance of microbiota in the gut of a model animal under various conditions, in accordance with one or more embodiments of the present disclosure;

FIG. 12A and 12B is a graph illustrating a pathway analysis of gut colonizing microbial strains in a model animal, in accordance with one or more embodiments of the present disclosure;

FIG. 12B is a graph illustrating a pathway analysis of gut colonizing microbial strains in a model animal, in accordance with one or more embodiments of the present disclosure;

FIG. 13 is a graph illustrating the effect of Mix10 against multiple Salmonella serovars, in accordance with one or more embodiments of the present disclosure;

FIG. 14 is a graph illustrating the effect of cell-free supernatants on S. Typhimurium growth, in accordance with one or more embodiments of the present disclosure.

### DETAILED DESCRIPTION

Reference will now be made in detail to the subject matter disclosed, which is illustrated in the accompanying drawings.

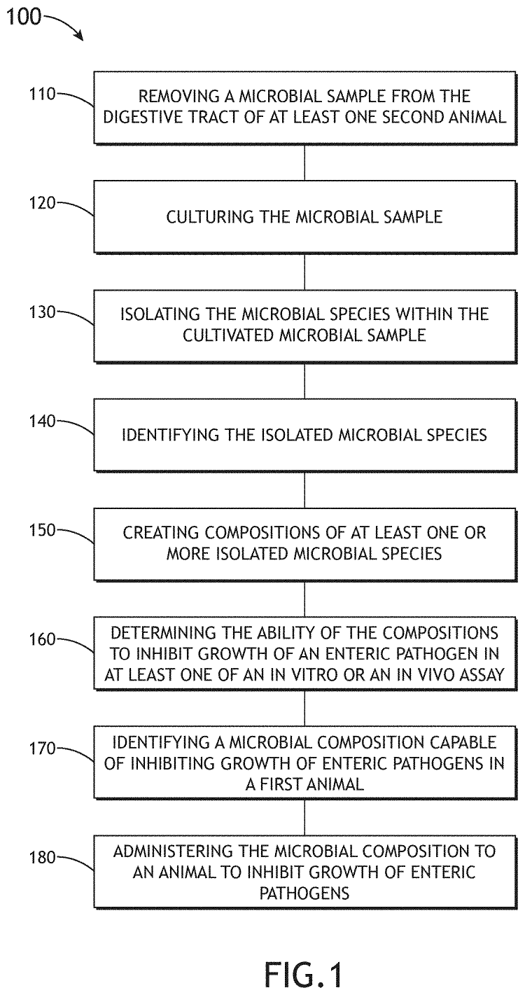

FIGS. 1-14 generally illustrate methods and compositions for inhibiting the colonization of enteric infections in animals, in accordance with one or more embodiments of the present disclosure.

Embodiments of the present disclosure are directed to methods and compositions for inhibiting the colonization of enteric infections in animals. The use of feces from healthy individuals enterically to treat sick individuals (e.g., fecal transplants) have been used in both human and non-human animals to treat various enteric infections. Fecal transplants allow the microbial biome of a healthy individual to infiltrate the gut of a sick individual, where the microbes from the biome may then outcompete pathogenic microbes within the gut for nutrients within various niches of the gut, resolving the pathogenic infection.

Fecal transplants are typically used on an individual basis (e.g., one donor to one recipient). For large populations of animals that are susceptible to outbreaks of enteric infection (e.g., poultry farms), large scale use of fecal transplants may not be not feasible. Also, the microbial composition of the fecal material is generally not known. Differences in the microbial composition of the fecal material between individual donors may result in some fecal material being effective in inhibiting and treating enteric infections, and some fecal material not being effective at all. Therefore, embodiments of the present disclosure are directed to methods for isolating and identifying microbial species within the fecal material of a healthy animal (e.g., a wild chicken known to be resistant to Salmonella infections). The isolated and identified species are then methodically combined into various compositions and tested to determine mixtures that are suited to inhibit pathogens that cause enteric infections (e.g., Salmonella). In this matter a probiotic with a well-defined mixture of microorganisms may be used to treat a variety of animals.

FIG. 1 illustrates a method 100 for identifying a microbial composition that inhibits the colonization of enteric infections in a first animal. The first animal is the animal to be treated for an enteric infection. The first animal may be any animal that can be treated for an enteric infection. In embodiments, the first animal is a bird. For example, the first animal may include, but is not limited to, a chicken, a turkey, a goose, or a duck.

In embodiments, the enteric pathogen may include any type of enteric pathogens known to cause an enteric disease, including, but not limited to, viruses, bacteria (e.g., from the phyla Firmicutes, Bacteroidetes, Actinobacteria, and Proteobacteria), fungi, protists, archaea, and multicellular parasites. For example, the enteric pathogen may be Salmonella Typhimurium (from the phylum Proteobacteria).

In embodiments, the method also includes a microbial composition that inhibits colonization of enteric pathogens. The microbial composition may take the form of any type of composition commonly used for entry into the digestive tract of an animal. For instance, the microbial composition may be a powder that is dissolved in liquid for the animal to drink. The microbial composition may also be formed as a capsule, a microcapsule, or a granular form for the animal to eat. The microbial composition may be a suppository or other type of formulation for use rectally. Alternatively, the microbial composition may be a liquid that is injected into the digestive tract of an animal (e.g., inoculating an embryonic chick).

In embodiments, the method 100 includes a step 110 of removing a sample from the digestive tract of a second animal. The second animal may be any animal that may be used as a source for therapeutic microbiota. In embodiments, the second animal is a bird (e.g., chicken, turkey, goose, duck, or other poultry). In embodiments, the second animal is a feral animal. It is recognized herein that feral animals may possess microbiomes that are more resistant to enteric pathogens than domesticated animals. Microbe-containing samples taken from the digestive tract of a feral animal likely contains microbes that inhibit the growth of enteric pathogens. Alternatively, the second animal may be a domesticated animal.

In embodiments, the method 100 includes a step 120 of culturing the microbial sample. The culture medium used for culturing the microbial sample may be any type of growth media known in the art for growing microbes, including, LB broth, blood agar, chocolate agar, brain heart infusion media, and the like. For example, the culture media may be a modified brain heart infusion media (BHI-M)

Culturing the microbial sample also involves control of environmental conditions (e.g., temperature, gas content). For example, the temperature for culturing the microbial sample may be the temperature of the gut of the second animal (e.g., 35° C. to 42° C.). For instance, the temperature of the culture may be approximately 37° C. In another instance, the temperature of the culture may be room temperature (e.g., 20° C. to 25° C.). The culture may be grown in an anaerobic or low oxygen environment. The culture may also be grown in an open atmosphere environment.

In embodiments, an iterative antibiotic supplementation is used to suppress bacteria that dominates the culture medium. The antibiotics used in the iterative antibiotic supplementation include any antibiotics known to suppress the growth of bacteria, including, but not limited to, gentamycin, kanamycin, neomycin, sulfamethoxazole, clindamycin, ampicillin, erythromycin, vancomycin, chloramphenicol, metronidazole, colistin, and the like. In embodiments, any mixture of antibiotics may be used in the iterative antibiotic supplementation. The iterative antibiotic supplementation may also include a heat treatment step.

In embodiments, the method 100 includes a step 130 of isolating the microbial species in the cultivated microbial sample. Isolating microbial species may involve plating of the cultivated microbial sample, resulting in the growth of individual colonies. Alternatively, the microbial species may be isolated through serial dilutions of the microbial sample.

In embodiments, the method 100 includes a step 140 of identifying the microbial species within the cultivated microbial sample. Identification of microbial species may include any method known in the art for identifying microbes, including genomic methods, proteomic methods, biochemical methods, and the like. Genomic methods for identifying microbial species include any methods known in the art for identifying microbial species, including, but not limited to, ribosomal RNA sequencing (e.g., 16S rRNA, 18S rRNA, or 28S rRNA), gene specific sequencing (e.g., rpoB, tuf, gyrA, gyrB or sodA), loop-mediated isothermal amplification assay, and microarray. Ribosomal RNA and gene specific sequences may be generated using any sequencing technology in the art, including, but not limited to, traditional slab sequencing, Illumine sequencing, 454 pyrosequencing, and the like.

Proteomic methods for identifying microbes include any proteomic methods capable of identifying of identifying microbes, including, but not limited to, MALDI-TOF MS, tandem mass spectrometry, and peptide sequencing. Biochemical methods may include the use of specific stains (e.g., Gram, acid-fast), antibody detection, and probe hybridization (e.g., FISH).

In embodiments, the method 100 includes a step 150 of creating compositions of at least one or more isolated microbial species. The selection of an isolated microbial species in a microbial composition may depend on the ability of the microbial species to inhibit growth of the enteric pathogen in vitro or in vivo. The selection of microbial species may also depend on the previously known abilities of mixtures of various microbial species to inhibit enteric pathogens.

In embodiments, the method 100 includes a step 160 of determining the ability of the composition to inhibit growth of an enteric pathogen in at least one of an in vitro or in vivo assay. In vitro determination of microbial compositions includes co-culture assays, where both the microbial composition and the enteric pathogen are cultured together in liquid media. After an incubation period, the broth is serially diluted and plated on agar plates. After incubation, the number of colony forming units (CFUs) are assessed.

In vivo determination of microbial composition includes testing the ability of the microbial composition to inhibit growth of enteric pathogens in an animal. The animal used for testing microbial compositions may include any model animal that is relevant for testing. For example, for identifying microbial compositions effective in chickens, the model animal is a newly hatched chicken. In this in vivo test, the hatchings are fed both the microbial composition and the enteric pathogen. After an incubation period, the hatchling is examined for the presence of the enteric pathogen and damage caused by the enteric pathogen. In the in vivo test, the animal may be gnotobiotic, having no flora within the digestive tract. Alternatively, an animal possessing flora within the digestive tract may be used.

In embodiments, the method 100 includes a step 170 of identifying a microbial composition capable of inhibiting growth of enteric pathogens in a first animal. The microbial composition may include any microorganism that has been identified to inhibit growth of an enteric pathogen. Microorganisms capable of inhibiting enteric pathogens (Salmonella Typhimurium) are listed herein and include representatives of the genera Faecalicoccus, Lactobacillus, Megamonas, Staphylococcus, Bacillus, Enterococcus, Olsenella, Megasphaera, Pseudoflavonifractor, or Massiliomicrobiota. Finally, in embodiments, the method 100 includes the step 180 of administering the microbial composition to an animal to inhibit growth of enteric pathogens.

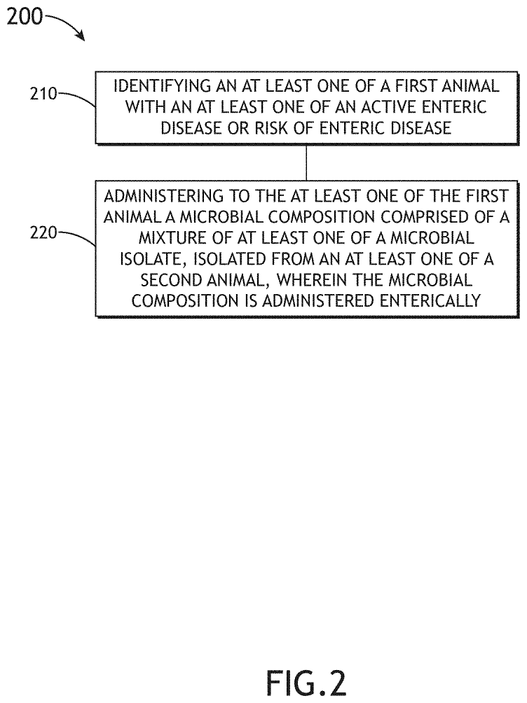

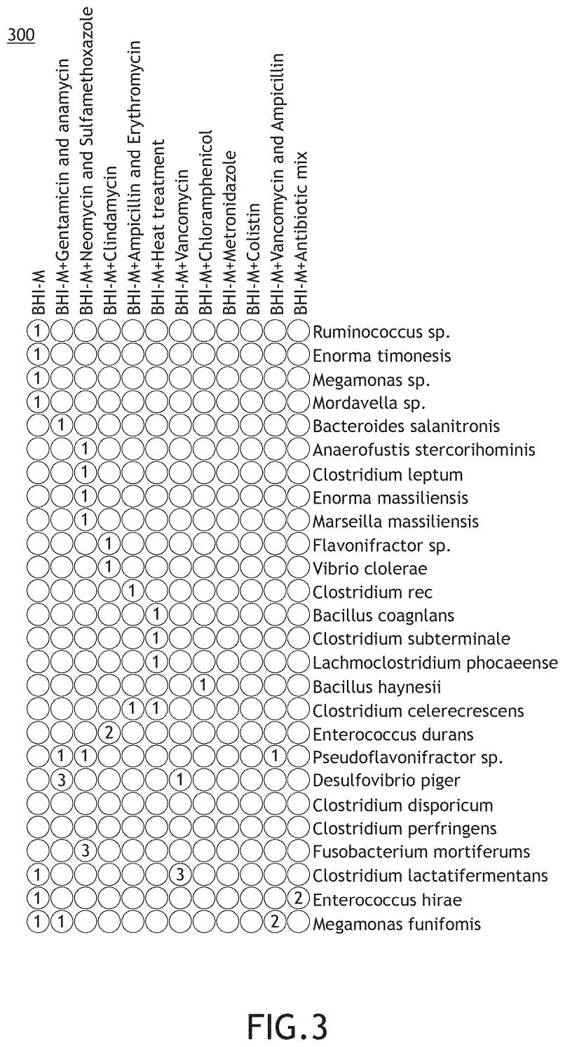

FIG. 2 illustrates a method 200 of administering a microbial composition that inhibits colonization of an enteric pathogen in animals. In a step 210 of the method 200, an animal at risk for enteric disease is identified. Animals at risk for enteric disease may include, but are not limited to, very young or very old animals, as well as animals with depressed immune systems (e.g., sick and injured animals). High-density populations of animals and animals that have lived in low-diversity microbial environments (e.g., factory farms) are also risk factors for enteric disease. Alternatively, animals that are presenting symptoms of enteric disease may also be identified for treatment.

In embodiments, the method 200 includes a step 220 of administering the microbial composition to a first animal. The administration of the microbial composition may be of any route of administration commonly used in the art for administration of probiotics, including, but not limited to, enteric administration (e.g., oral, rectal). Enteric administration includes any method of delivering a therapeutic substance into the digestive tract of the subject, including, but not limited to, eating, drinking, administering through a nasogastric tube, administering through the rectum (e.g., enema, suppository), and direct injection into the digestive tract of an animal). In embodiments, the microbial composition may comprise any form known in the art capable of being administered to an animal, including, but not limited to, a pill, a tablet, a solution, a suspension, an enema, and a suppository.

Embodiments of the present disclosure are directed to a microbial composition that inhibits the colonization of an enteric pathogen (e.g., Salmonella) in an animal. In embodiments, the microbial composition is prepared by a process that includes a number of steps. The first step to prepare the microbial composition is to remove a microbial sample from the digestive tract of an animal. In some aspects, the animal is feral. Another step is to prepare the microbial composition is to culture the microbial sample. In embodiments, the culture of microbial sample involves iterative antibiotic supplementation to suppress growth of dominating microbes in culture. The preparation of the microbial composition includes a step of isolating the microbial species within the cultivated microbial sample. The preparation of the microbial sample further includes the identification of the isolated microbial species. The methods for identification of isolated microbial species are described herein.

In embodiments, the preparation of the microbial composition includes a step of creating compositions of at least one or more microbial species. The preparation of the microbial composition includes a step of testing the microbial compositions to determine the ability of the compositions to inhibit growth of an enteric pathogen in vitro or in vitro. Methods for the testing of the microbial compositions are described herein. The microbial compositions are also tested on an animal to determine whether the microbial composition is capable of inhibiting the growth of enteric pathogens. Finally, in embodiments, the preparation of the microbial composition includes a step of fashioning the microbial composition into a form capable of enteric composition (e.g., a pill, enema, or oral solution).

In embodiments, the microbial composition comprises a therapeutically effective amount of a plurality of viable microorganisms from one or more genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition only includes microbes from Faecalicoccus, Lactobacillus, Megamonas, Staphylococcus, Bacillus, Enterococcus, Olsenella, Megasphaera, Pseudoflavonifractor, and Massiliomicrobiota genera.

In embodiments, the microbial composition comprises a therapeutically effective amount of a plurality of viable microorganisms from one or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota

In embodiments, the microbial composition comprises a therapeutically effective amount of a plurality of viable microorganisms from two or more genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition only includes microbes from Faecalicoccus, Lactobacillus, Megamonas, Staphylococcus, Bacillus, Enterococcus, Olsenella, Megasphaera, Pseudoflavonifractor, and Massiliomicrobiota genera.

In embodiments, the microbial composition comprises a therapeutically effective amount of a plurality of viable microorganisms from two or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota

In embodiments, the microbial composition comprises a therapeutically effective amount of a plurality of viable microorganisms from three or more genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition only includes microbes from Faecalicoccus, Lactobacillus, Megamonas, Staphylococcus, Bacillus, Enterococcus, Olsenella, Megasphaera, Pseudoflavonifractor, and Massiliomicrobiota genera.

In embodiments, the microbial composition comprises a therapeutically effective amount of a plurality of viable microorganisms from three or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota

In embodiments, the microbial composition comprises a therapeutically effective amount of a plurality of viable microorganisms from four or more genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition only includes microbes from Faecalicoccus, Lactobacillus, Megamonas, Staphylococcus, Bacillus, Enterococcus, Olsenella, Megasphaera, Pseudoflavonifractor, and Massiliomicrobiota genera.

In embodiments, the microbial composition comprises a therapeutically effective amount of a plurality of viable microorganisms from four or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota.

In embodiments, the microbial composition comprises a therapeutically effective amount of a plurality of viable microorganisms from five or more genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition only includes microbes from Faecalicoccus, Lactobacillus, Megamonas, Staphylococcus, Bacillus, Enterococcus, Olsenella, Megasphaera, Pseudoflavonifractor, and Massiliomicrobiota genera.

In embodiments, the microbial composition comprises a therapeutically effective amount of a plurality of viable microorganisms from five or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota.

In embodiments, the microbial composition comprises a therapeutically effective amount of a plurality of viable microorganisms from six or more genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition only includes microbes from Faecalicoccus, Lactobacillus, Megamonas, Staphylococcus, Bacillus, Enterococcus, Olsenella, Megasphaera, Pseudoflavonifractor, and Massiliomicrobiota genera.

In embodiments, the microbial composition comprises a therapeutically effective amount of a plurality of viable microorganisms from six or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota.

In embodiments, the microbial composition comprises a therapeutically effective amount of a plurality of viable microorganisms from seven or more genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition only includes microbes from Faecalicoccus, Lactobacillus, Megamonas, Staphylococcus, Bacillus, Enterococcus, Olsenella, Megasphaera, Pseudoflavonifractor, and Massiliomicrobiota genera.

In embodiments, the microbial composition comprises a therapeutically effective amount of a plurality of viable microorganisms from seven or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota.

In embodiments, the microbial composition comprises a therapeutically effective amount of a plurality of viable microorganisms from eight or more genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition only includes microbes from Faecalicoccus, Lactobacillus, Megamonas, Staphylococcus, Bacillus, Enterococcus, Olsenella, Megasphaera, Pseudoflavonifractor, and Massiliomicrobiota genera.

In embodiments, the microbial composition comprises a therapeutically effective amount of a plurality of viable microorganisms from eight or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota.

In embodiments, the microbial composition comprises a therapeutically effective amount of a plurality of viable microorganisms from genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota.

In embodiments, the microbial composition comprises a therapeutically effective amount of a plurality of viable microorganisms from genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota. In this regard, the microbial composition may include microbes from Faecalicoccus, Lactobacillus, Megamonas, Staphylococcus, Bacillus, Enterococcus, Olsenella, Megasphaera, Pseudoflavonifractor, and Massiliomicrobiota genera, as well as microbes from one or more genera that is not Faecalicoccus, Lactobacillus, Megamonas, Staphylococcus, Bacillus, Enterococcus, Olsenella, Megasphaera, Pseudoflavonifractor, and Massiliomicrobiota.

In embodiments, the microbial composition comprises a therapeutically effective amount of a plurality of viable microorganisms from one or more genera or species selected from a group consisting of Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition only includes microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from one or more genera or species selected from a group comprising Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition may include microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor, as well as microbes from one or more species of genera that is not Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from two or more genera or species selected from a group consisting of Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition only includes microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from two or more genera or species selected from a group comprising Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition may include microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor, as well as microbes from one or more species of genera that is not Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from three or more genera or species selected from a group consisting of Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition only includes microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from three or more genera or species selected from a group comprising Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition may include microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor, as well as microbes from one or more species of genera that is not Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from four or more genera or species selected from a group consisting of Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition only includes microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from four or more genera or species selected from a group comprising Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition may include microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor, as well as microbes from one or more species of genera that is not Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from five or more genera or species selected from a group consisting of Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition only includes microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from five or more genera or species selected from a group comprising Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition may include microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor, as well as microbes from one or more species of genera that is not Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from six or more genera or species selected from a group consisting of Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor. In this regard, the microbial composition only includes microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor.

In embodiments, the microbial composition comprises a therapeutically effective amount of a plurality of viable microorganisms from six or more genera or species selected from a group comprising Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition may include microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor, as well as microbes from one or more species of genera that is not Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor.

In embodiments, the microbial composition comprises a therapeutically effective amount of a plurality of viable microorganisms from seven or more genera or species selected from a group consisting of Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition only includes microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from seven or more genera or species selected from a group comprising Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition may include microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor, as well as microbes from one or more species of genera that is not Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from eight or more genera or species selected from a group consisting of Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition only includes microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudo flavonifractor.

In embodiments, the microbial composition comprises a therapeutically effective amount of a plurality of viable microorganisms from eight or more genera or species selected from a group comprising Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition may include microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor, as well as microbes from one or more species of genera that is not Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor.

In embodiments, the microbial composition comprises a therapeutically effective amount of a plurality of viable microorganisms from genera or species selected from a group consisting of Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition only includes microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor.

In embodiments, the microbial composition comprises a therapeutically effective amount of a plurality of viable microorganisms from genera or species selected from a group comprising Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor. In this regard, the microbial composition may include microbes from Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor, as well as microbes from one or more species of genera that is not Faecalicoccus pleomorphus, Lactobacillus agilis, Staphylococcus saprophyticus, Bacillus paralicheniformis, Enterococcus durans, Megasphaera statonii, Megamonas funiformus, Massiliomicrobiota timonensis, Olsenella, and Pseudoflavonifractor.

It should be understood that the microbial compositions listed above are intended to inhibit colonization of an enteric pathogen (e.g., Salmonella) in an animal (e.g., a chicken).

In embodiments, the microbial composition may contain relatively equal ratios of each microorganisms. For example, for a composition comprising Faecalicoccus pleomorphus and Lactobacillus agilis, the composition may contain a 1:1 ratio of Faecalicoccus pleomorphus microorganisms to Lactobacillus agilis microorganisms. In embodiments, the microbial composition may contain unequal ratios of each microorganisms. For example, for a composition comprising Faecalicoccus pleomorphus and Lactobacillus agilis, the composition may contain a 1:100 ratio of Faecalicoccus pleomorphus microorganisms to Lactobacillus agilis microorganisms.

In embodiments, one or more microbes in the microbial composition may contain living organisms that are in culture (e.g., not dormant, such as in a spore). Alternatively, in embodiments, one or more microbes in the microbial composition may contain living organisms that are dormant (e.g., a spore).

It should be noted that a first animal may include one animal, or may include multiple animals. Likewise, a second animal may include one animal, or may include multiple animals. As mentioned herein, the first animal and/or second animal may be poultry (e.g., a chicken). It should also be noted that the first animal and second animal may be the same species or belong to different species. For example, both the first animal and the second animal may be a chicken. In another example, the first animal may be a chicken, and the second animal may be a sheep.

The following examples are intended only to further illustrate the invention and are not intended to limit the scope of the subject matter which is defined by the claims.

### EXAMPLE 1

### Development of the Feral Chicken Gut Microbiota Library

For the isolation of bacteria from the feral chicken gut, six intestinal samples were pooled. The pooled intestinal sample was serially diluted and was plated on modified Brain Heart Infusion agar (BHI-M) with 12 different selective conditions. The modified Brain Heart Infusion agar (BHI-M) contained the following ingredients: 37 g/L of BHI, 5 g/L of yeast extract, 1 ml of 1 mg/mL menadione, 0.3 g L-cysteine, 1 mL of 0.25 mg/L of resazurin, 1 mL of 0.5 mg/mL hemin, 10 mL of vitamin and mineral mixture, 1.7 mLof 30 mM acetic acid, 2 mL of 8 mM propionic acid, 2 mL of 4 mM butyric acid, 100 pl of 1 mM isovaleric acid, and 1% of pectin and inulin. All cultures were performed inside an anaerobic chamber (Coy Laboratories) containing 5% CO2, 10% H2, and 85% N2 maintained at 37° C. A total of 1,300 colonies was picked from all conditions and dilutions. Selected colonies were streaked on base BHI-M agar, and a single colony was selected for preparing stocks and species identification.

Species identity of the isolates was determined using Matrix-Assisted Laser Desorption/Ionization-Time of Flight (MALDI-TOF) or 16S rRNA gene sequencing. For MALDI-TOF identification, individual colonies were smeared on the MALDI-TOF target plate and lysed by 70% formic acid. MALDI-TOF targets were covered with 1 mL of a matrix solution. MALDI-TOF was performed through the Microflex LT system (Bruker Daltonics). AMALDI-TOF score >1.9 was considered as positive species identification. Isolates that could not be speciated at this cut-off were identified using 16S rRNA gene sequencing. The growth of each bacterial species measured following overnight incubation in BHI-M using a spectrophotometer at OD₆₀₀. Thereafter, stocks were maintained by adjusting the OD to 0.5. Aerotolerance of the strains was tested by culturing in aerobic, anaerobic and microaerophilic conditions. To this end, individual strains were first cultured overnight in BHI-M broth at 37° C. under anaerobic condition. The optical density at 600 nm (OD₆₀₀) of the cultures was adjusted to 0.5. Then, 1% of OD₆₀₀ adjusted cultures were inoculated in fresh BHI-M media in triplicates. Each replicate of cultures was then incubated under anaerobic, microaerophilic and aerobic conditions. For microaerophilic condition, a hypoxic box was used to incubate the culture. After 24 hours of incubation, the growth of individual bacteria was determined by measuring OD_{600.}

### EXAMPLE 2

### Co-culture Assays and Formulation of the Bacterial Blends

A co-culture assay was used to screen all bacterial species for S. Typhimurium inhibition capacity. In this assay, each species was anaerobically cultured together with S. Typhimurium in a ratio of 9:1 in 1.0 ml of BHI-M broth and incubated at 37° C. for 24 h. To quantify the magnitude of S. Typhimurium inhibition by each species, the individual co-cultures were 10-fold serially diluted with 1X anaerobic phosphate buffer saline (PBS) and plated on Xylose Lysine Tergitol 4 (XLT4) agar (BD Difco, Houston, Tex.). The plates were incubated aerobically at 37° C. for 24 hours followed by plating on XLT4 agar and colony forming units (CFU) were enumerated to determine the degree of S. Typhimurium inhibition.

### EXAMPLE 3

### Determination of in vivo Effect of Ten Species Consortium using Gnotobiotic Chicken Model

A gnotobiotic chicken model was used to determine the in vivo effect of probiotics. Fertile Specific Pathogen Free (SPF) eggs were wiped with Sporicidin® disinfectant solution (Contec®, Inc.), an FDA approved sterilizing solution, followed by washing in sterile water. Further, the eggs were incubated at 37° C. and 55% humidity for 19 days. Eggs containing an embryo, confirmed after candling, were dipped in Sporicidin® for 15 s and wiped with sterile water before transferring to a sterile gnotobiotic isolator maintained at 37° C. and 65% humidity until hatching. Chickens were fed with 107 CFU of probiotic at day three, four and five post-hatching, followed by 105 CFU of S. Typhimurium challenge on day six post-hatching. Chickens were euthanized by cervical dislocation on day two and day five post-infection. The cecum contents and tissues were aseptically collected and stored at −80° C. S. Typhimurium load in the cecum contents were determined by plating on Salmonella selective XLT4 agar.

### EXAMPLE 4

### Histopathology

The tissues for histopathology were initially fixed in 10% Formalin. The cecum tissues were trimmed and processed into paraffin blocks by routine histopathological methods, i.e., gradual dehydration through a series of ethanol immersion, followed by xylene and then paraffin wax. They were sectioned at 4pm and stained with hematoxylin and eosin (HE), followed by scanning of glass slides in a Philips scanner. Further, the cecum pathology was evaluated based on scores. Representative gastrointestinal tissues and liver were examined in a pilot study. Lesions were graded in liver, base and body of cecum, colon, and proximal and distal ileum. A score of 0 was given for no visible lesions; 1 for inflammatory cell infiltrates in tissues; and 2 for exudation of fibrin and inflammatory cells into the lumen of the intestine, for all regions examined. The scores for cecum were chosen for publication, since all culture work was performed on cecal isolates.

### EXAMPLE 5

Assessment of Immune Response using Quantitative Reverse-Transcriptase (Q-PCR)

To study the chicken antibacterial response after bacterial treatment, immune pathways related to antibacterial immune response in the cecal tissue was determined. Total RNA from cecal tissue samples was extracted using the TRIzol® reagent (Ambion RNA, Invitrogen) method. Briefly, an average weight of 0.042 g of cecal tissue per sample (n=7 per group) was used. Tissue samples from each group were pooled and homogenized separately in TRIzol® reagent (Ambion|RNA, Invitrogen) (1 mL per 100 mg of tissue sample) and extraction was performed according to manufacturer's protocol. RNA concentration was determined from spectrophotometric optical density measurement (A260/A280) using NanoDrop™ One (Thermo Fisher Scientific, Wilmington, Del.). For Q-PCR, cDNA was synthesized using First-strand cDNA synthesis kit (New England BioLabs, Inc.) according to the manufacturer's protocol. To get enough cDNA for downstream procedures, 4 μg RNA was used as input in a cDNA synthesis reaction.

The dynamics of the chicken antibacterial response was analyzed using RT2 Profiler PCR Array (cat# 148ZA-12, Qiagen) according to the manufacturer's protocol. Real-time RT-PCR was performed following the manufacturer's protocol using an ABI 7500 HT thermal cycler (Applied Biosystems). A cycle threshold cut-off of 0.2 was applied to all gene amplifications and was normalized to Ribosomal protein L4 (RPL4) and Hydroxymethylbilane synthase (HMBS) as they were stably expressed across all treatment groups from a panel of 5 housekeeping genes. The normalization and further analysis of the data were performed at the Data analysis center, Qiagen. A bar graph of fold change in the expression of major cytokines, TLRs (Toll-like receptors) and other immune factors was generated using GraphPad Prism software (GraphPad Software, USA).

### EXAMPLE 6

### Determination of the Population Structure of the Bacterial Consortium in the Cecum using 16S Amplicon Analysis:

The relative abundance of individual species in the probiotic after colonizing the gnotobiotic chicken were determined using 16S amplicon sequencing. Genomic DNA from cecal contents was extracted using the PowerSoil DNA isolation kit (Mo Bio Laboratories Inc, Calif.). To ensure even lysis of the microbial community, bead beating was performed on 100 mg of cecal contents for 10 min using a TissueLyser (Qiagen, Germantown, Md.). Remaining steps for DNA isolation were performed as per manufacturer's instruction. Final elution of DNA was carried out in 50 μL nuclease-free water. The quality of DNA was assessed using a NanoDrop One™ (Thermo Fisher Scientific, Wilmington, Del.) and quantified using a Qubit Fluorometer 3.0 (Invitrogen, Carlsbad, Calif.). The samples were stored at −20° C. until further use. DNA yield of four samples (2nd day) and two samples (5th day) from the Salmonella alone treated group was low for further procedures and were removed from the downstream processes. The enrichment of the microbial DNA was performed using the NEBNext Microbiome DNA Enrichment Kit (New England Biolabs Inc, Mass.) according to the manufacturer's instruction.

A total of 31 DNA samples were used for 16S rRNA gene sequencing using the Illumina MiSeq platform with 250 base paired-end V2 chemistry. DNA library preparation was performed using Illumina Nextera XT library preparation kit (Illumina Inc. San Diego, Calif.) targeting the V3 and V4 region of the 16S rRNA gene sequence. The amplicons were then purified using Agencourt AMPure XP beads (Beckman Coulter). Before loading, libraries were bead normalized and pooled in equal concentration.

CLC Genomics Workbench (version 11.0.1) (Qiagen) was used to analyze the 16S rRNA sequence data. An average of 72,749 raw reads per sample (ranging from 34,962 to 100,936) was imported to CLC workbench. After the initial quality check, reads with low Q30 score were removed by trimming with a quality score limit of 0.01. Paired reads were merged at a minimum alignment score of 40. OTU clustering was performed at the 97% similarity level using a locally downloaded Greengenes database and a custom database of full-length 16S rRNA gene sequence of probiotic species. Best matches were found at chimera cross over cost of 3 and kmer size of 6. Finally, on an average 28759 reads per sample were used to generate OTUs. A total of 80 OTUs generated in this analysis were then aggregated at the genus level. The abundance table and metadata were then used in Calypso software to create stacked bar plots. Total sum normalization (TSS) was used to normalize the datasets by dividing feature read counts by the total number of reads in each sample. The plot was generated using only those OTUs (genus level) that have more than 0.5 percent relative abundance across all samples.

Genome analysis of probiotic species using next-generation sequencing. The bacterial DNA kit (D3350-02, eZNA™, OMEGA bio-tek, USA) was used to isolate the genomic DNA for next-generation sequencing. The quality of DNA was assessed using Qubit Fluorometer 3.0 (Invitrogen, Carlsbad, Calif.). The sequencing was performed using Illumine MiSeq sequencer with MiSeq Reagent Kit v3 (2×300 base paired-end chemistry). The reads were assembled using Unicycler that builds an initial assembly graph from short reads using the de novo assembler SPAdes 3.11.1. The quality assessment for the assemblies was performed using QUAST. The open reading frames (ORFs) were predicted using Prodigal 2.6 in the Prokka software package. To determine the functional modules in the genome, the amino acid sequences were mapped against the KEGG (Kyoto Encyclopedia of Genes and Genomes) database using the BlastKOALA genome annotation tool. Each KEGG module was represented on a scale of 0 to 4 (0=complete, 1=1 block missing, 2=2 block missing and 3=module absent). The matrix was used for hierarchical clustering using the MORPHEUS server provided by the Broad Institute for constructing the heat map using Pearson correlation matrix and average linking method. As mentioned previously, the strains of culture library were isolated from the pooled intestinal content of six feral chickens. This sample (inoculum) was used for DNA isolation, sequencing and analysis for our previous study. In this study, the assembled contigs from this inoculum were used to predict the putative protein coding sequences using FragGeneScan. The resulting amino acid sequences were clustered using CD-HIT to reduce the sequence redundancy. The clustered proteins were then annotated against the KEGG Orthology (KO) database to assign the molecular functions using GhostKOALA (PMID: 26585406). The complete modules present in the metagenomics sample were compared against the colonized (n=7) and non-colonized strains (n=3). The heat map was constructed using Pearson correlation matrix and average linking method against the Morpheus server.

### EXAMPLE 7

### Results: Development of the Feral Chicken Gut Microbiota Library.

It is known that microbiota from healthy adult chicken could inhibit the growth of S. enterica in the gut. It was reasoned that because of higher microbial exposure, feral chicken would have more diverse gut microbiome than commercial chicken and a high percentage of the microbiota in the feral chicken gut could have inhibitory capacity against S. enterica. To ascertain this, a bacterial library from feral chicken cecal contents using anaerobic culture conditions was isolated. A modified Brain Heart Infusion was used as the base culture medium which is hereafter referred to as BHI-M.

When a non-selective medium is used for cultivation, it is common that fast-growing bacteria use up space and nutrients in the medium. To avoid this problem, an iterative antibiotic supplementation of BHI-M to suppress bacteria that dominated the base medium was used. For example, from the base BHI-M, when 32 bacterial species were isolated, five species (Massiliomicrobiota timonensis, Faecalicoccus pleomorphus, Eubacterium cylindroides, Collinsella sp., and Olsonella sp.) accounted for 52.6% of colonies. To suppress the growth of these species, BHI-M with gentamycin and kanamycin which allowed isolation of several species that were not isolated from the plain medium was supplemented. Using twelve such selection conditions, 1,300 isolates were selected. Species identity of 1,023 isolates was determined by either MALDI-TOF or 16S rRNA gene sequencing.

FIG. 3 is a chart 300 illustrating an overview of the culture conditions as well as diversity and frequency of isolated microbial species in an example microbial composition, in accordance with one or more embodiments of the present disclosure. Intestinal content of six feral chickens was pooled, stocked, and cultured using 12 culture combinations. Species identification was performed using matrix-assisted laser desorption/ionization-time of flight (MALDI-TOF) or 16S rRNA sequencing. A numerical heat map showed diversity and abundance of bacterial species in a culture library was generated using Morpheus, versatile matrix visualization and analysis software. The numbers in each circle represent the frequency of isolation of that species. Overall, 51 species were identified which belong to phyla Firmicutes (36 species), Bacteroides (five species), Proteobacteria (five species), Actinobacteria (four species) and Fusobacterium (one specie). When 97.82% 16S rRNA sequence identity is applied as species delimiter, the culturing approach also identified eleven previously uncultured species that are candidates to be designated as type strains.

### EXAMPLE 8

### Results: Screening and the Selection of Defined Bacterial Consortium that Inhibits Salmonella.

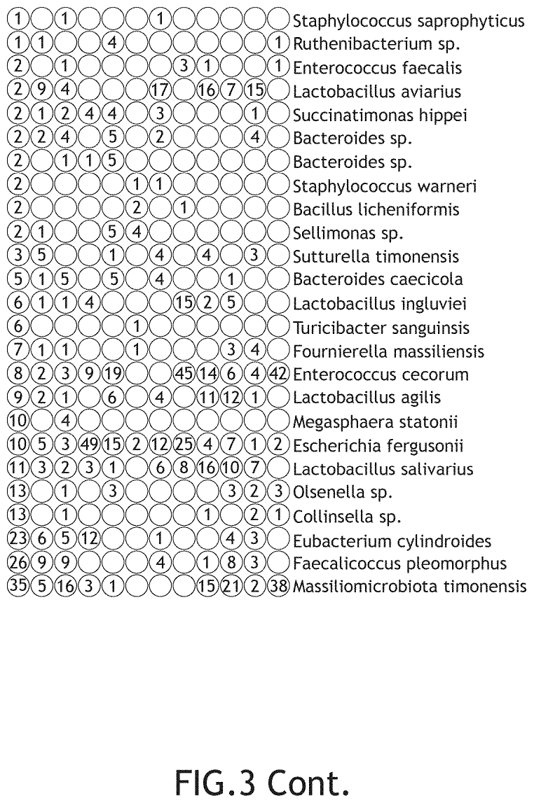

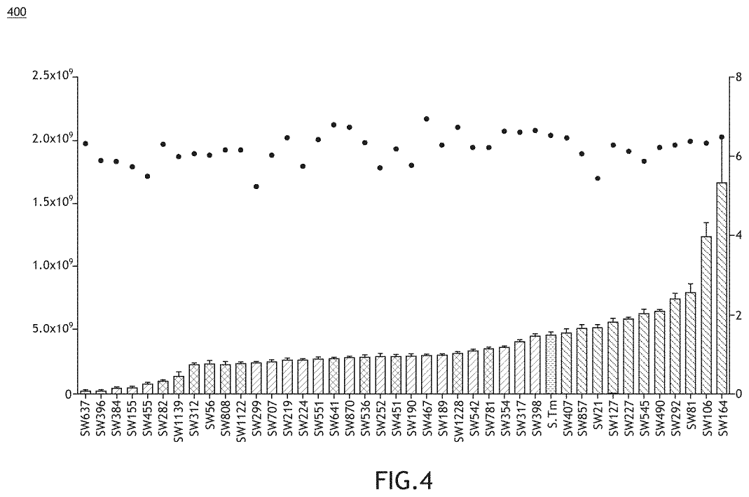

To determine the species that could inhibit Salmonella in the library, using a co-culture assay, the inhibition capacity of species representative isolates against S. Typhimurium was tested. FIG. 4 is a graph 400 illustrating microbial species that show varying degrees of inhibition against S. Typhimurium, in accordance with one or more embodiments of the present disclosure. Forty-one species isolated from the pool cecum of feral chickens were used for co-culture assays in this experiment. The OD₆₀₀ of overnight bacterial culture was adjusted to 0.5 and individual strains were mixed with S. Typhimurium at a ratio of 9:1. The CFU of Salmonella (left y-axis) and pH (right y-axis) were determined after 24 hours incubation. S. Typhimurium growth enhancing strains (e.g., those presenting Salmonella CFUs of 5.0×10⁹ or greater, such as SW164) are grouped to the right. S. Typhimurium growth inhibiting strains (e.g., those presenting Salmonella CFUs of less than 5.0×10⁹, such as SW637) grouped to the left. Twelve S. Typhimurium inhibiting strains were chosen to generate 66 combinations containing 10 species.

As shown in graph 400 in FIG. 4, from the total collection, 30 species showed varying degrees of inhibition against S. Typhimurium. Since the reduction in pH during bacterial growth is inhibitory to S. Typhimurium, it was determined whether pH was reduced at the end of the co-culture assay. Estimation of pH showed that it ranged between 5.5 and 7.0. In the majority of the cases, pH did not drop below 6.0. This may mean that the inhibition of S. Typhimurium by these strains may not be primarily mediated by the production of organic acids that would have lowered the pH of the medium. Interestingly, this screen also showed that eleven species in the collection enhanced the growth of S. Typhimurium (FIG. 4).

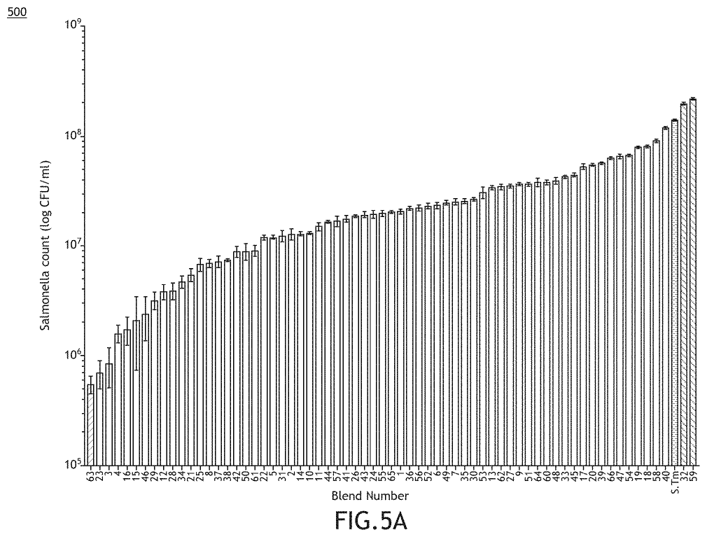

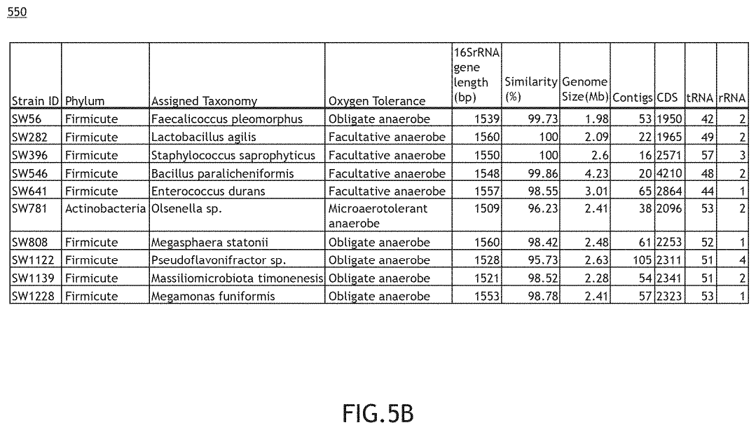

Further, it was tested whether the Salmonella inhibition capacity of the strains are improved if a subset of strains is pooled together. To reduce the complexity of the pool, twelve inhibitory bacterial strains that are fast growing and maintaining a pH above 5.8, were selected to formulate the blend. Since there is the chance that species composition of the blend may positively or negatively influence the S. Typhimurium inhibition capacity, several subsets were made using a combinatorial approach in which two strains are randomly removed from the 12 species blend. With this combinatorial approach, 66 different combinations composed of 10 species could be formulated. The inhibitory capacity of all these blends using co-culture assay were then tested. FIG. 5A is a bar graph 500 illustrating the effectiveness of various microbial blends for S. Typhimurium inhibition, in accordance with one or more embodiments of the present disclosure. As shown in bar graph 500 in FIG. 5, the blend approach improved the S. Typhimurium inhibition. Out of 66 combinations, blend 63 (e.g., the bar furthest to the left) showed the highest inhibition with 2 log reduction of S. Typhimurium compared to control. While the majority of the blends were inhibitory in varying degrees, blend number 32 and 59 increased the growth of S. Typhimurium. This was unexpected because all the strains selected were individually inhibiting Salmonella. It is an indication that the community composition of the bacterial blends can override the individual strain phenotype (Salmonella inhibition in this case). Therefore, combinatorial testing can reveal gut microbiota sub-community that might produce an entirely different phenotype than that of the individual species membership in a bacterial consortium.

Since blend number 63 showed the highest inhibition of Salmonella among all combinations tested, this blend was used for further in vivo experiments. This blend, which hereafter referred to as Mix10, was composed of Faecalicoccus pleomorphus, Lactobacillus agilis, Staphylococcus saprophyticus, Bacillus paralicheniformis, Enterococcus durans, Olsenella sp., Megasphaera statonii, Pseudoflavonifractor sp., and Massiliomicrobiota timonensis. Based on 16S rRNA gene similarity search against EzTaxon and NCBI databases, two strains (Olsenella sp. and Pseudoflavonifractorsp.) in this blend represented novel species of their respective genera. These results are consistent with the reasoning that feral chicken gut harbors diversity that includes new taxa that are inhibitory against Salmonella. FIG. 5B illustrates a table 550 describing the bacterial strains used to formulate MIX10, in accordance with one or more embodiments of the present disclosure.

### EXAMPLE 9

### Results: Mix10 consortium confers partial protection against S. Typhimurium infection.

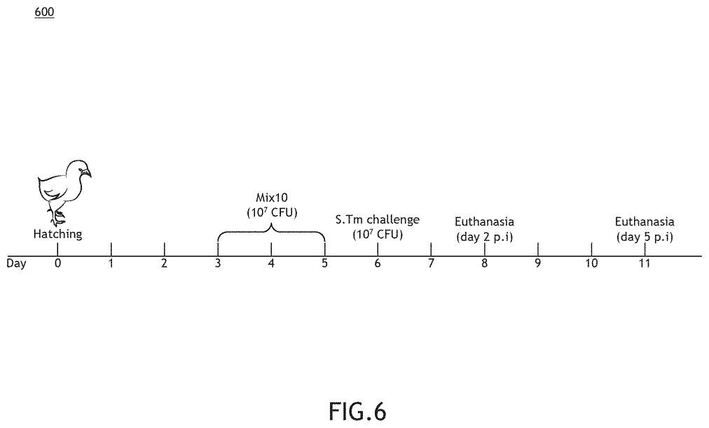

The effect of Mix10 colonization on host health and in vivo inhibition capacity was determined. To this end, a gnotobiotic chicken (Gallus gallus) model previously developed was used. FIG. 6 is a chart 600 illustrating a detailed timeline for testing microbial blends for S. Typhimurium inhibition in vivo, in accordance with one or more embodiments of the present disclosure. At hatching day, the gnotobiotic chickens were divided into four different groups representing gnotobiotic chicken (GNO), gnotobiotic chicken with S. Typhimurium infection (S.Tm), Mix10-colonized gnotobiotic chicken with S. Typhimurium infection (Mix10+S.Tm) and Mix10-colonized gnotobiotic chicken (Mix10). Additionally, one group represented conventional chicken (CON) with Mix10 inoculation and S. Typhimurium infection. Mix10 at 10⁷ CFU was administered via oral drenching at day 3, 4 and 5 post-hatching. Chickens were challenged with 10⁵ S. Typhimurium CFU.

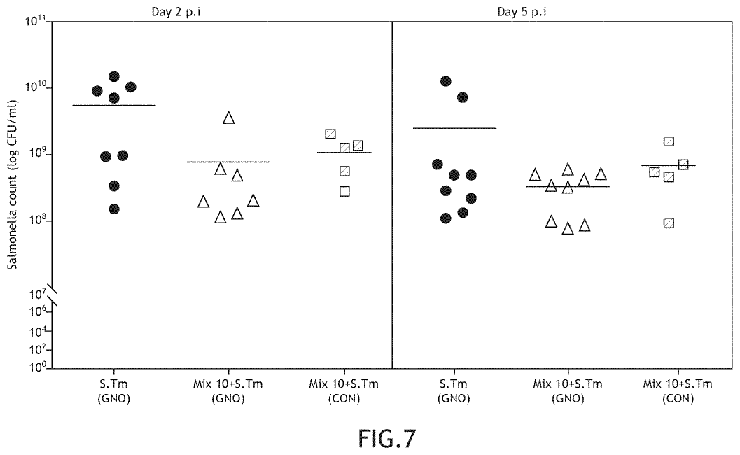

FIG. 7 is a graph 700 illustrating the inhibition of a microbial blend on S. Typhimurium in vivo, in accordance with one or more embodiments of the present disclosure. Half the number of chickens in each group were euthanized at day two post-infection, and others on day five post-infection. Salmonella load was determined from the cecum content. The load of S. Typhimurium in gnotobiotic chicken colonized with Mix10 and challenged with S. Typhimurium on day two and five post inoculation were 7.7×10⁸CFU/ml and 3.4×10⁸ CFU/ml, respectively, as shown in graph 700. In the S. Typhimurium infected group, the Salmonella CFU on day two and five post-infection were 5.5×10⁹ CFU/ml and 2.6×10⁹ CFU/ml, respectively. When compared to this group, the Salmonella load was reduced sevenfold in the group colonized with Mix10 and challenged with S. Typhimurium. Reduction of Salmonella load in the Mix10 colonized group is in line with the expectation that this consortium could inhibit Salmonella in vivo.

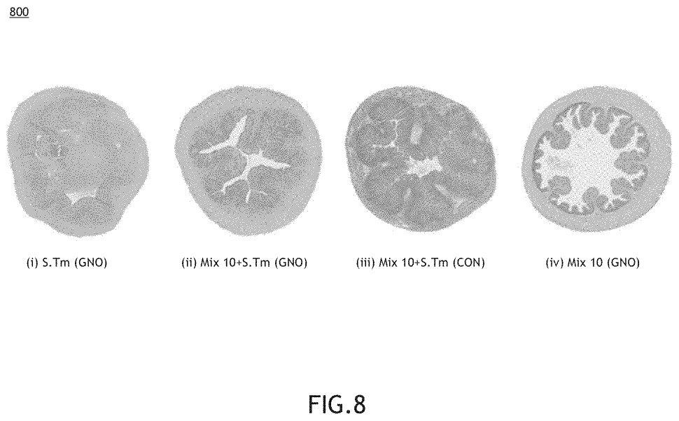

The effect of Mix10 colonization on intestinal physiology was examined via histopathology. Inflammatory lesions of cecal tissues were determined using histological sections, as shown in the photo 800 in FIG. 8. Fibrinopurulent exudate was observed in the lumen of S. Typhimurium infected group. Also, the mucosa was swollen due to mixed inflammatory cell infiltrates such as macrophages, lymphocytes, and heterophils in lamina propria. Erosion of mucosa was evident with the loss of mucosal folds (FIG. 8; S. Tm). Under higher magnification, early transmural inflammation with minimal peritonitis was observed. However, in the Mix10 colonized and S. Typhimurium infected group, the severity of the infection was reduced. Inflammation of the mucosa was still detected which narrowed the luminal space. Mucosal folds were noticeable but inflammatory cells were still spotted. The mucosa was not eroded, and no exudate was found in the lumen.

Similar evidences were observed in Mix10-colonized conventional chickens with S. Typhimurium infection. However, a small amount of exudate is noted in the lumen. In this group, there was also submucosal and transmural edema with macrophages and heterophils. Gnotobiotic chickens with Mix10 inoculation showed a large empty lumen with a small amount of ingesta. Thin mucosa with mucosal folds was protruding into the lumen. Mild cellularity of lamina propria with scattered glands was observed. When these histopathological figures were compared, mucosal inflammation was very high with S. Typhimurium infection but less intense with Mix10 inoculation.

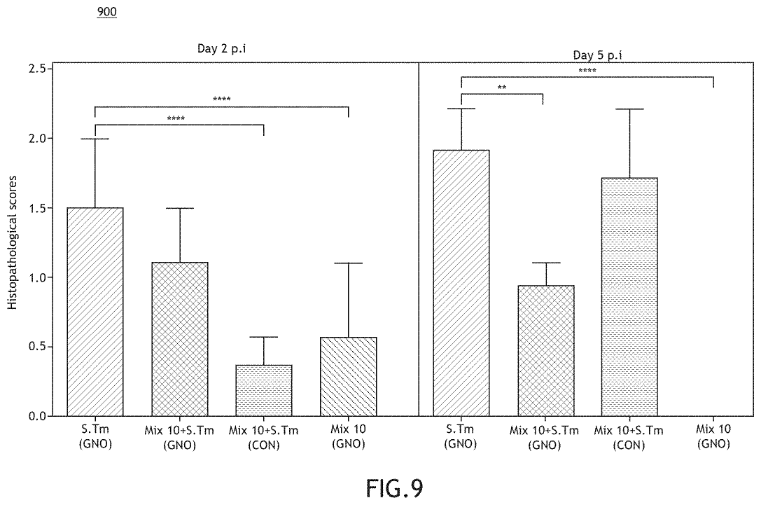

Mix10 resulted in the fewest lesions as depicted by the histopathological scores compared to S. Typhimurium infection, as shown in graph 900 in FIG. 9. The chickens in Mix10 colonization and S. Typhimurium infection group showed significantly lower histopathology scores compared to chickens in S. Typhimurium infection group at day two post-infection. Furthermore, gnotobiotic chickens infected with S. Typhimurium presented increased histopathology scores at day five post-infection while the scores were reduced in chickens inoculated with Mix10 and S. Typhimurium infection. Also, it should be noted that Mix10 had no noticeable effect on the mucosa. The results indicated that Mix10 may normalize chicken gut by supporting the development of intestinal tissue and reducing inflammatory symptoms and reducing mucosal damage during S. Typhimurium infection. The significant difference scoring code for FIG. 9 is as follows: (P<0.05); *P<0.05, **P<0.01, ***P<0.001, and ****P<0.0001.

### EXAMPLE 10

### Mix10 Modulates Gut Immunity and Reduces Salmonella-induced Inflammation.

In chicken, Salmonella infection is known to trigger inflammation of the gut by the production of pro-inflammatory cytokines such as interleukin (IL)-1 and IL-6, chemokines such as IL-8, and type 1 T helper (Th1) cell cytokines such as IL-2 and interferon-γ (INF-γ), along with a cascade of other cytokines including tumor necrosis factor-α (TNF-α), IL-12, IL-15 and IL-18. To determine whether Mix10 colonization could ameliorate Salmonella-induced inflammation, the expression level of 96 inflammation-associated genes were measured by quantitative RT-PCR (Q-PCR) array in the chicken caecum.

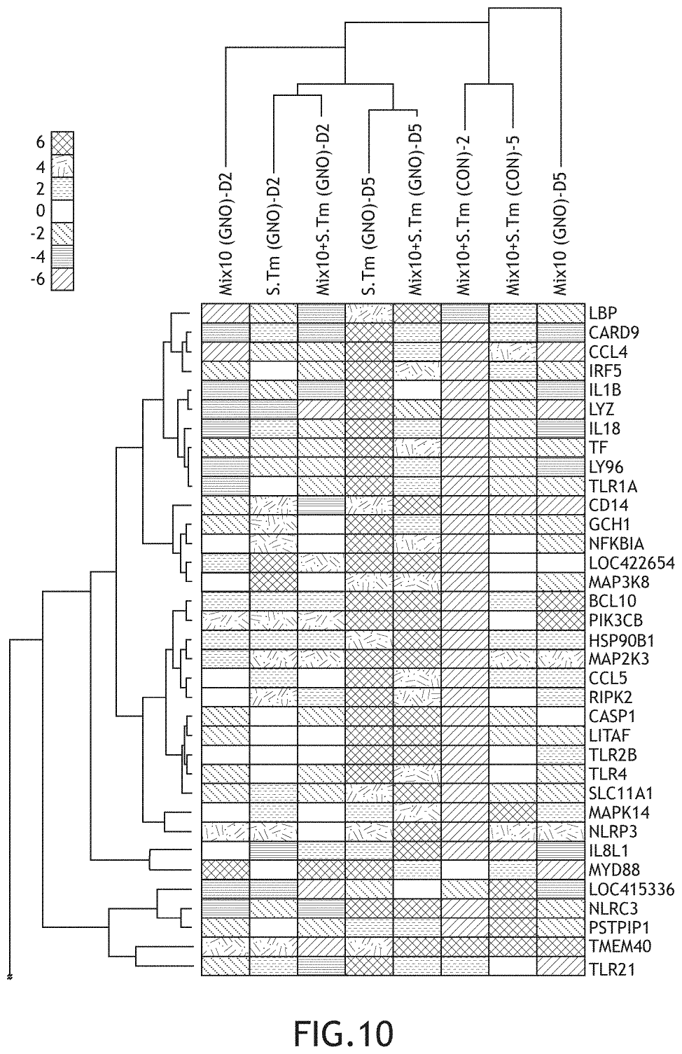

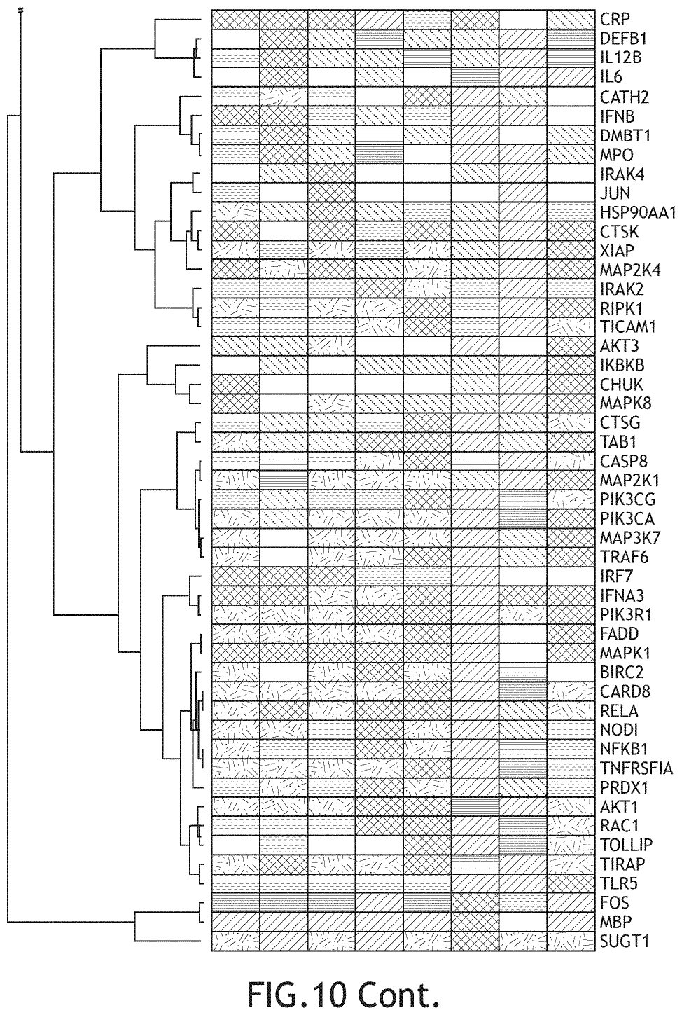

FIG. 10 is a graph 1000 illustrating an mRNA profile of pooled cecal tissue for various inflammatory cytokines, chemokines and other genes under various conditions, in accordance with one or more embodiments of the present disclosure. Total RNA from pooled cecal tissue of each group was used to perform a real-time qRT-PCR with a single PCR comprised of 84 pathway/disease/functionally related genes and five housekeeping genes. A heat map presented the relative expression of 84 genes associated immune responses was generated using Morpheus, versatile matrix visualization and analysis software. Data was clustered using Pearson correlation with complete method within Morpheus package. Fold change in expression comparing to gnotobiotic chicken control of S. Typhimurium infection in gnotobiotic chickens, S. Typhimurium infection in Mix10-colonized gnotobiotic chickens, Mix10-colonized gnotobiotic chickens and Mix10-colonized conventional chickens infected with S. Typhimurium at day 2 and 5 post-infection.

As expected, chickens infected with S. Typhimurium showed multiple fold increase in the expression levels of various pro-inflammatory cytokines and chemokines; IL-18, IL-113, IL-6 and IL-8L1 at day two and five post-infection. Massive expression of these pro-inflammatory cytokines were correlated to upregulated expression of other genes such as Toll-like receptors (TLRs), Nucleotide-binding oligomerization domain containing 1 (NOD₁), Myeloid differentiation primary response gene 88 (MyD88) which are cell surface pattern receptor recognitions (PPRs) and activators of inflammatory pathways suggesting capability of S. Typhimurium in the induction of inflammation in microbiota-free chickens as compared to gnotobiotic chickens, as shown in graph 1000 in FIG. 10. Furthermore, the chickens colonized with Mix10 did not generate a severe inflammatory response as the expression levels of pro-inflammatory cytokines (IL-6 and IL-18) was comparatively low. Antimicrobial peptides (AMPs) are crucial for eliminating a broad range of pathogens through pathogen-associated molecule pattern (PAMP) receptors. Two major AMPs; cathelicidin2 (CATH2) as well as defensin-beta 1 (DEFB1) have been reported in chicken. In this study, chickens colonized with Mix10 and challenged with S. Typhimurium showed a higher level of CATH2 as well as DEFB1 when compared to the group infected with S. Typhimurium (FIG. 10). The results suggested that colonization of Mix10 species in the chicken gut can ameliorate S. Typhimurium induced inflammation by activating AMPs and anti-inflammatory immune response.

### EXAMPLE 11

### Results: Mix10 in vivo Community Composition and Functional Genomic Analysis.

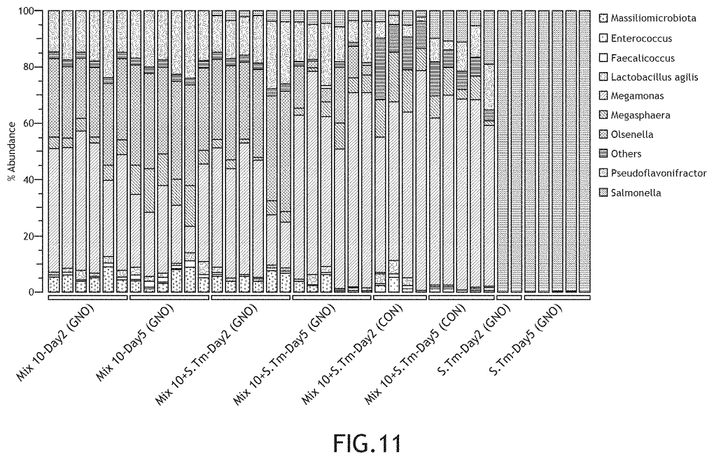

Although all species in Mix10 in equal proportion were inoculated in gnotobiotic chicken, it is most likely that some species would reach high abundance while others may have low abundance or do not colonize the gut at all. To ascertain this, 16S rRNA amplicon-based microbiome profiling of the cecal samples were performed. FIG. 11 is a graph 1100 illustrating the relative abundance of microbiota in the gut of a model animal under various conditions, in accordance with one or more embodiments of the present disclosure. The OTU clustering was performed at 97% similarity level using CLC Genomics Workbench (version 11.0.1) with the Greengene database and a custom database of full length 16S rRNA gene sequences of Mix10 and Salmonella. The stacked bar plots of relative abundance at genus and species level was generated using Explicet software tool (version 2.10.5).

These results, as shown in graph 1100 in FIG. 11, indeed showed the domination of some species while some showed low abundance and no colonization of two species. Olsenella, Pseudoflavonifractor, and Megamonas together constituted more than 70% of the Mix10 population in the chicken cecum. Three species; S. saprophyticus, B. paralicheniformis, and E. durans were below 0.5% normalized read cutoff, indicating poor or no colonization. Olsenella, Pseudoflavonifratctor, and Megamonas dominated in the Mix10 alone, and Mix10+Salmonella challenged group. The abundance of Salmonella in this group when compared Salmonella alone inoculated group was substantially lower (FIG. 11). This matches well with the several fold reduction of Salmonella determined from the same samples by CFU enumeration (FIG.7). Since Olsenella, Pseudoflavonifratctor, and Megamonas dominated in all groups and substantially reduced Salmonella in the chicken cecum, it is reasonable to think that these three species contributed the majority of the in vivo effect observed, including normalization of the gut, reduction of inflammation and exclusion of Salmonella.

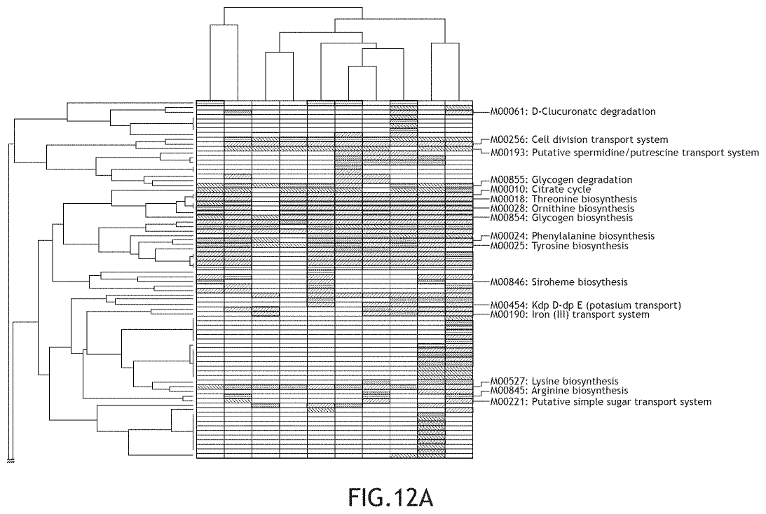

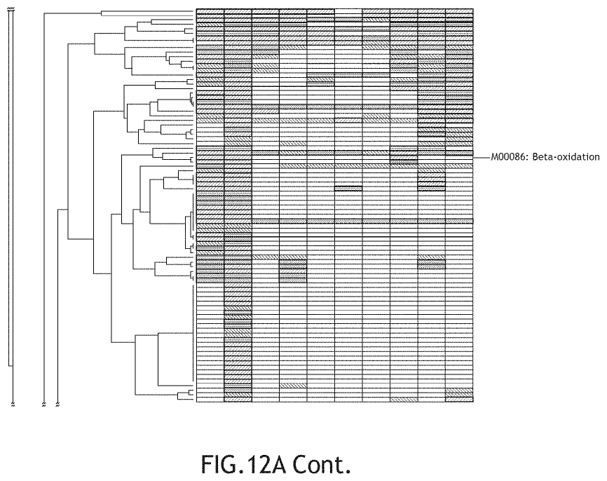

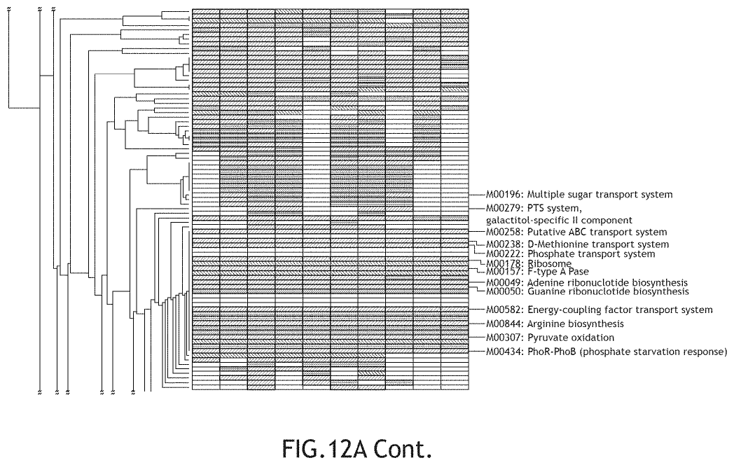

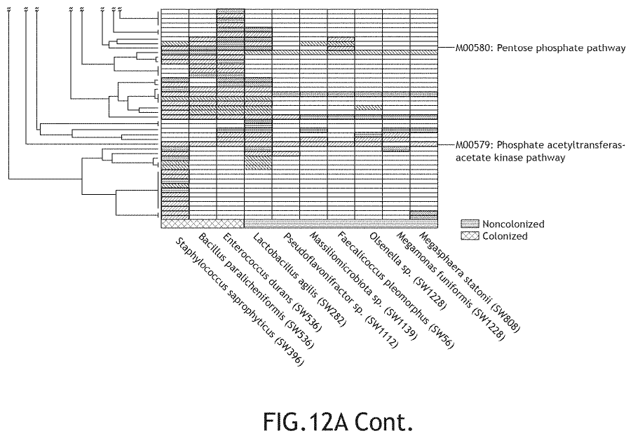

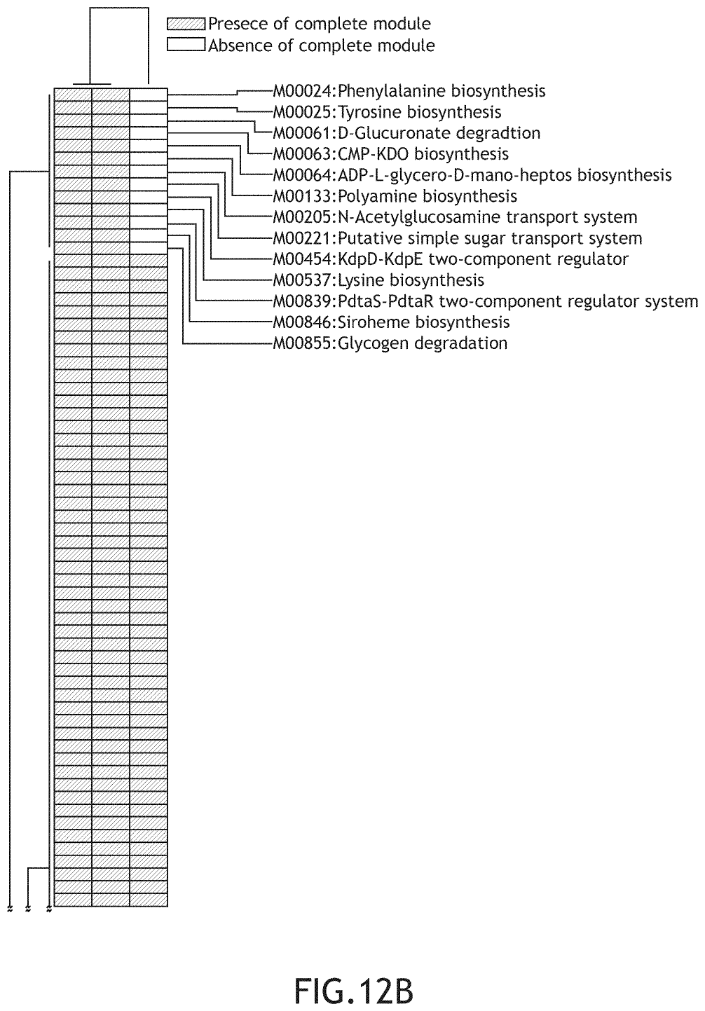

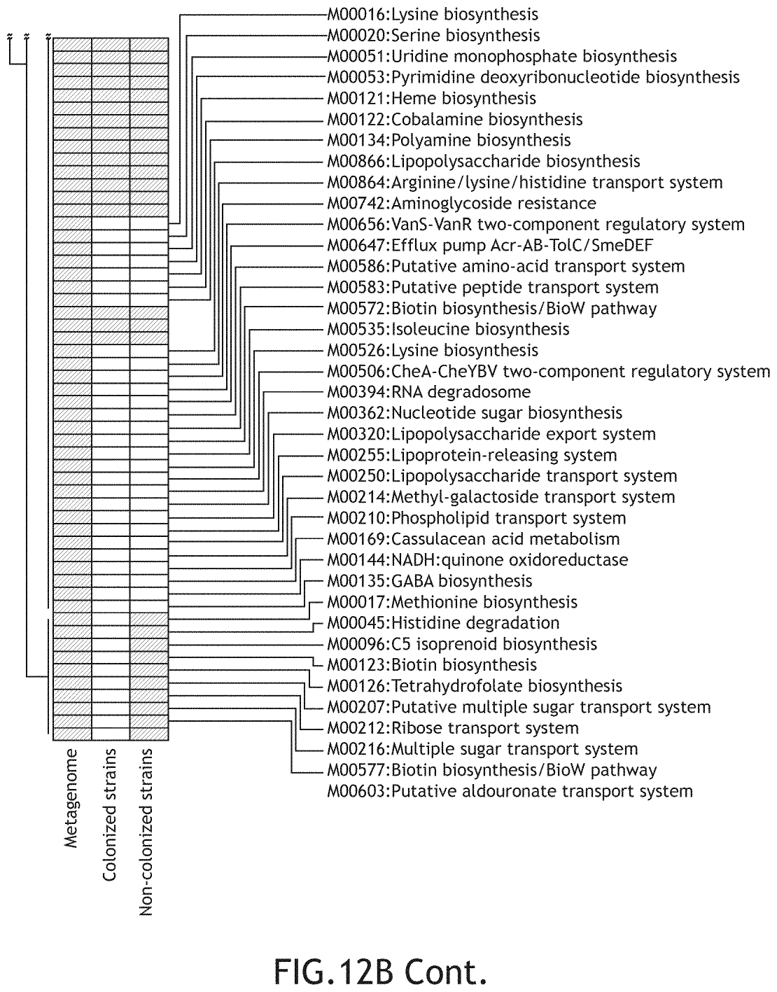

To decipher the overall functional capabilities of the members of Mix10, the genomes were sequenced and analyzed. Since the presence of functional modules computed using KEGG has been used to design defined gut bacterial blends that partially inhibited Salmonella, the presence of KEGG modules correlated with in vivo colonization of strain in the study was examined. FIG. 12A and FIG. 12B are graphs 1200, 1250 illustrating a pathway analysis of gut colonizing microbial strains in a model animal, in accordance with one or more embodiments of the present disclosure.

The presence and completeness of KEGG modules in the strains were annotated and used for hierarchical clustering (Pearson correlation). A total of 293 KEGG modules were present either completely or partially across 10 species of Mix10 (FIG. 12A). Based on the results from amplicon sequencing, only 7 organisms; Olsenella, Pseudoflavonifratctor, Megamonas, Megasphaera, Massiliomicrobiota, Faecalicoccus and Lactobacillus were able to colonize the chicken gut. However, out of 293 modules detected across all strains, 243 modules with 159 complete modules were contributed by Bacillus, Enterococcus and Staphylococcus which did not colonize the chicken gut. This indicates that presence of KEGG modules in the genome of Mix10 species may not be the primary determinant of colonization ability in the chicken gut. This was further evident when all functional modules in Mix10 was compared against the predicted complete modules in the feral chicken fecal metagenome as shown in graph 1250 in FIG. 12B. Although colonized strains clustered closer to metagenome of the feral chicken microbiome, presence or absence of KEGG module did not reveal any clear partitioning in this comparison.

### EXAMPLE 12

### Results: Mix10 Reduces Growth of Multiple Salmonella Serovars that are Dominant in Poultry

To determine the range of inhibitory activity of Mix10, the inhibitory activity against members of other dominant serotypes was examined. Additional 4 serovars, S. Typhimurium (monophasic), S. Heidelberg, S. Infantis and S. Enteritidis that are dominant in poultry according to CDC reports were used in this experiment. A co-culture assay was performed as previously described.

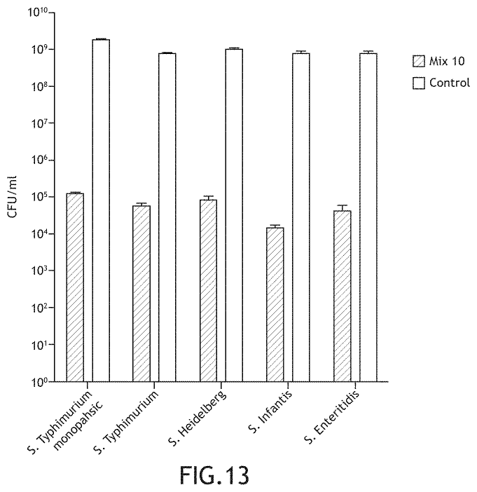

FIG. 13 is a graph 1300 illustrating the effect of Mix10 against multiple Salmonella serovars, in accordance with one or more embodiments of the present disclosure. The graph illustrates the CFU/ml of 5 serotypes of Salmonella after 24 h of co-culture with Mix10 and Salmonella monoculture. The result exhibits the significant reduction of Salmonella in co-culture compared to control in all serovars. In addition, Mix10 had same level of inhibitory activity against other dominant serovars indicating that Mix10 is a representative sub-community which broadly inhibit different serovars of S. enterica.

### EXAMPLE 13

### Results: The Inhibitory Effect of Mix10 on S. Typhimurium is not Due to the Action of Secreted Proteins

To investigate whether proteinaceous is the mechanism of Salmonella exclusion by Mix10, cell-free supernatant co-culture assay was performed. Overnight anaerobic growth of individual species was collected and pooled together at equal amount. The cell pellets were removed by centrifugation at 3,000 rpm for 1 h. The supernatant was then filtered through 0.4 μm pore size. The purified supernatant was adjusted pH to 6.5-6.8 using NaOH and HCl. The supernatant was divided into 3 conditions; no treatment, heat treatment and proteinase K treatment. For heat treatment, the supernatant was heated at 100° C. for 1 h. For proteinase K treatment, the supernatant was incubated in 50 μg/mlof proteinase K for 1 h at 37° C. Then, 40 μl of OD₆₀₀ 0.5 S. Typhimurium was cultured in 50% supernatant in 1 ml of fresh BHI-M. Two-fold media dilution with 1X PBS was used to culture Salmonella as a control sample. After 24 hour of incubation, Salmonella cells was enumerated as previously described.

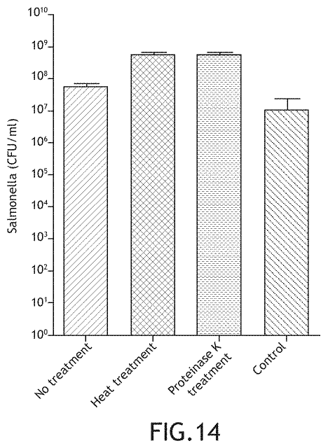

FIG. 14 illustrates a graph 1400 showing the effect of cell-free supernatants on S. Typhimurium growth in accordance with one or more embodiments of the present disclosure. None of cell-free supernatants was able to reduce S. Typhimurium in co-culture assay as in Mix10 co-culture assay. The supernatant with no treatment increased S. Typhimurium compared to control. When secreted protein molecules in supernatant were degraded by heat and proteinase K, S. Typhimurium growth was improved. This indicated that the mechanism contributed to Salmonella exclusion in this study was nutrient competition and not secreted proteins by Mix10.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

It should be understood that this disclosure is not limited to the particular methodology, protocols, and reagents, etc., described herein and as such may vary. The terminology used herein is for the purpose of describing particular embodiments only, and is not intended to limit the scope of the present disclosure. It is also to be understood that embodiments of the methods disclosed herein may include one or more of the steps described herein. Further, such steps may be carried out in any desired order and two or more of the steps may be carried out simultaneously with one another. Two or more of the steps disclosed herein may be combined in a single step, and in embodiments, one or more of the steps may be carried out as two or more sub-steps. Further, other steps or sub-steps may be carried in addition to, or as substitutes to one or more of the steps disclosed herein.

A number of embodiments of the invention have been described. Nevertheless, it will be understood that various modifications may be made without departing from the spirit and scope of the disclosure. Accordingly, other embodiments are within the scope of the following claims.

What is claimed:

1. A method for identifying a microbial composition that inhibits colonization of an enteric pathogen in at least one first animal comprising:
removing a microbial sample from a digestive tract of at least one second animal;
culturing the microbial sample;
isolating a microbial species within a cultivated microbial sample;
identifying the microbial species;
creating compositions of one or more isolated microbial species;
determining an ability of the compositions to inhibit growth of an enteric pathogen in at least one of an in vitro or an in vivo assay; and
identifying a microbial composition capable of inhibiting growth of enteric pathogens in the at least one first animal.

2. The method of claim 1, further comprising administering the microbial composition to one or more of the at least one first animals to inhibit growth of enteric pathogens.

3. The method of claim 1, wherein the at least one first animal is poultry.

4. The method of claim 1, wherein the at least one second animal is feral.

5. The method of claim 1, wherein an iterative antibiotic supplementation is used to suppress growth of dominating microbes in culture.

6. The method of claim 1, wherein the enteric pathogen comprises Salmonella.

7. The method of claim 1, wherein the composition comprises a therapeutically effective amount of a plurality of viable microorganisms from one or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota.

8. A microbial composition that inhibits colonization of an enteric pathogen in at least one first animal, prepared by a process comprising the steps of:
removing a microbial sample from a digestive tract of at least one second animal;
culturing the microbial sample;
isolating a microbial species within a cultivated microbial sample;
identifying the microbial species;
creating a composition of at least one or more isolated microbial species;
determining an ability of the composition to inhibit growth of an enteric pathogen in at least one of an in vitro or in vivo assay;
identifying a microbial composition capable of inhibiting growth of enteric pathogens in the at least one first animal; and
fashioning the microbial composition into a form capable of enteric administration.

9. The microbial composition of claim 8, wherein the microbial composition comprises a therapeutically effective amount of a plurality of viable microorganisms from one or more genera selected from a group comprising of Faecalicoccus, Lactobacillus, Megamonas, Staphylococcus, Bacillus, Enterococcus, Olsenella, Megasphaera, Pseudo flavonifractor, and Massiliomicrobiota.

10. The microbial composition of claim 8, wherein the enteric pathogen comprises Salmonella.

11. The microbial composition of claim 8, wherein the at least one first animal is poultry.

12. A method of administering a microbial composition that inhibits colonization of an enteric pathogen in at least one animal, comprising:
identifying an at least one first animal with an at least one of an active enteric disease or risk of enteric disease,
administering to one or more of the at least one first animal a microbial composition comprised of a mixture of at least one of a microbial isolate, isolated from an at least one second animal, wherein the microbial composition is administered enterically.

13. The method of claim 12, wherein the microbial composition comprises a therapeutically effective amount of a plurality of viable microorganisms from one or more genera selected from a group comprising Faecalicoccus, Lactobacillus, Megamonas, Staphylococcus, Bacillus, Enterococcus, Olsenella, Megasphaera, Pseudoflavonifractor, and Massiliomicrobiota.

14. The method of claim 12, wherein the enteric pathogen comprises Salmonella.

15. The method of claim 12, wherein the at least one first animal is poultry.

16. A microbial composition that inhibits colonization of an enteric pathogen in at least one animal comprising, a therapeutically effective amount of a plurality of viable microorganisms from two or more genera selected from a group consisting of Faecalicoccus, Lactobacillus, Megamonas, Staphylococcus, Bacillus, Enterococcus, Olsenella, Megasphaera, Pseudoflavonifractor, and Massiliomicrobiota.

17. The microbial composition of claim 16, wherein the plurality of viable microorganisms comprise two or more species or genera selected from the group consisting of Faecalicoccus pleomorphus, Lactobacillus agilis, Staphylococcus saprophyticus, Bacillus paralicheniformis, Megamonas funiformus Enterococcus durans, Megasphaera statonii, Massiliomicrobiota timonensis, Olsenella, and Pseudoflavonifractor.

18. The microbial composition of claim 16, wherein the enteric pathogen comprises Salmonella.

19. The microbial composition of claim 16, wherein the at least one animal is poultry.

20. The microbial composition of claim 16, wherein the microbial composition is formed as at least one of a capsule, a microcapsule, or a granular form.
